(19) United States
(12) Patent Application Publication
(10) Pub. No.: US 2023/0310289 A1
(43) Pub. Date: Oct. 5, 2023

(54) Personal Care Compositions and Methods

(71) Applicant: Colgate-Palmolive Company, New York, NY (US)

(72) Inventors: Alejandro JIMENEZ, Mexico City (MX); Mariana Martinez HEREDIA, Delegación Miguel Hidalgo (MX); Sharon KENNEDY, Randallstown, MD (US); Juan Mauricio MORA PALE, Mexico City (MX)

(73) Assignee: Colgate-Palmolive Company, New York, NY (US)

(21) Appl. No.: 17/629,520

(22) PCT Filed: Dec. 22, 2021
(86) PCT No.: PCT/US2021/064941

§ 371 Date: Jan. 24, 2022

Related U.S. Application Data
(60) Provisional application No. 63/129,337, filed on Dec. 22, 2020.

(51) Int. Cl. A61K 8/37; A61K 8/92; A61K 8/28; A61K 8/34; A61Q 15/00

(52) U.S. Cl. CPC A61K 8/375; A61K 8/922; A61K 8/28; A61K 8/342; A61Q 15/00; A61K 2800/30

(57) ABSTRACT

The application is directed to personal care products (e.g., deodorants and/or anti-perspirants) with improved delivery of aluminum (Al) active, wherein the personal care product of the disclosure contains low amounts of cyclomethicone, is substantially free of cylcomethicone, or there is no amount of cyclomethicone present. Methods of preparing the personal care composition and uses of the personal care composition are also disclosed.

### FIELD OF THE INVENTION

The application relates to personal care products (e.g., deodorants and/or anti-perspirants) with improved delivery of aluminum (Al) active, wherein the personal care products of the disclosure contain relatively low amounts of cyclomethicone, are substantially free of cylcomethicone, or there is no amount of cyclomethicone present. Methods of preparing the personal care composition and uses of the personal care composition are also disclosed.

### BACKGROUND

Underarm deodorants control odor by eliminating the bacteria that cause odor. Conventional antiperspirant salts, such as aluminum, aluminum, and/or zirconium salts, tend to be acidic in aqueous solution, a property which makes them effective bactericides, thereby providing a deodorant benefit, but which can also cause skin irritation. In addition, long-acting antiperspirant compositions typically contain increased amounts of active ingredients as a route to obtaining sustained effectiveness. At the same time, skin sensitivity and vulnerability to various compounds may limit the practical upper concentration in personal care formulations. It is now believed that up to 50% of the population has sensitive skin with a reduced irritation threshold.

Personal care compositions, such as antiperspirant or deodorant compositions and dual purpose antiperspirant-deodorant compositions, may be used to reduce body odor. Antiperspirant or deodorant compositions may be applied to axillary (underarm) regions to prevent or treat perspiration, limit the growth of odor-causing bacteria, or apply a fragrance. Antiperspirant or deodorant compositions may be delivered topically as roll-on, gel, cream, stick or aerosol formulations.

In recent years, there has been increasing regulatory pressures to reduce or remove certain emollients, such as cyclomethicone, from anti-perspirant or deodorant type products. Cyclomethicone refers to the group of methyl siloxanes or cyclic polydimethylsiloxane polymers with low viscosity and high volatility commonly used as skin emollients in personal care formulations. Cyclomethicone has been widely used, for example, because of the efficacy it has providing the stick a silky, dry and smooth finish without being oily. In addition, an antiperspirant product has to have the right balance to attach onto the skin for delivering the active (Al salts) into the pores for blocking sweat and in consequence provide the sweat reduction attribute. However, the applied product has to be easy to wash off when consumers take showers; this is a sensorial attribute that consumers demand from an anti-perspirant.

Replacing cylcomethicone, but at the same time maintaining the same desirable properties of an anti-perspirant or deodorant product, is a challenge within the personal care composition industry.

### BRIEF SUMMARY

In one aspect, the application is directed to improved delivery of aluminum (Al) active in personal care products (e.g., deodorants and/or anti-perspirants), wherein the personal care product contains relatively low amounts of cyclomethicone, substantially free of cylcomethicone, or no amount of cyclomethicone present. Without being bound by theory, the inclusion of a glyceryl-based emulsifier (e.g., glyceryl monostearate) acts to make the composition matrix less hydrophobic, which in turn improves diffusion of water to release a given active and deliver the active to a user's pores. This improved delivery allows for the use of less a metal active (e.g., aluminum zirconium tetrachlorohydrex active) while still maintaining efficacy.

In one embodiment, the personal care composition comprises glyceryl-based emulsifier and a metal containing antiperspirant agent/active, and either reduced amounts of cyclomethicone or is substantially free of cyclomethicone. In this embodiment, the delivery of active is unexpectedly improved, or at least at parity, with the performance of a composition that contains cyclomethicone.

In another embodiment, a method of delivering a metal containing antiperspirant agent/active provided by applying a composition comprising a glyceryl-based emulsifier and a metal containing antiperspirant agent/active.

In addition to antiperspirants and/or deodorants, the invention also encompasses other personal care compositions for application to the skin, for example hand soaps or body washes, comprising a potentially irritating active ingredient and/or precursors thereof. The invention further provides methods of reducing sweat comprising applying the composition to skin, and methods of killing bacteria comprising contacting the bacteria with the composition.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

Unless otherwise specified, all percentages and amounts expressed herein and elsewhere in the specification should be understood to refer to percentages by weight relative to the total composition. The amounts given are based on the active weight of the material.

### DETAILED DESCRIPTION

The following description of the preferred embodiment(s) is merely exemplary in nature and is in no way intended to limit the invention, its application, or uses.

The invention provides a personal care composition (Composition 1.0), which can be applied to the skin, as well as methods of reducing body odor and/or moisture comprising administering Composition 1.0 et seq to the skin.

In another aspect, the invention provides Composition 1.0 (e.g., a stick deodorant or antiperspirant) for application to the skin. In one aspect, Composition 1.0 is a personal care composition that comprises:
From 25%-45% by wt. of the total composition of a plant-based gelling agent (e.g., palm kernel oil) (e.g., 25%-38% by wt.) (e.g., 25%-41% by wt.)
From 15%-30% by wt. of the total composition of one or more structuring agents (e.g., a mixture of stearyl and cetyl alcohol) (e.g., a 70/30 Stearyl/Cetyl Alcohol Blend)
A glyceryl-based emulsifier (e.g., glyceryl monostearate or glyceryl monolaurate);
A metal containing antiperspirant agent (e.g., aluminum zirconium tetrachlorohydrex glycine);
A first source of emollients, wherein the emollient comprises one or more selected from: C12-C15 alkyl benzoate, mineral oil, caprylyl glycol, dimethicone, dicaprylyl ether, ethyl trisiloxane and combinations thereof;
   wherein the amount of the first source of emollients does not exceed 15% by wt of the total composition (e.g., from 0.05%-14.9% by wt of the total composition) (e.g., from 5%-14.9% by wt. of the total composition) (e.g., from 10%-14.9% by wt. of the total composition).
1.1 The personal care composition of Composition 1.0, wherein the plant-based gelling agent is selected from the group consisting of: waxes, esters of fatty acid and fatty alcohol, triglycerides, partially or fully hydrogenated soybean oil, partially or fully hydrogenated castor oil, other partial or fully hydrogenated plant oils, or other cosmetically acceptable materials (e.g., which are solid or semi solid at room temperature and provide a consistency suitable for application to the skin) and combinations thereof.
1.2 The personal care composition of Composition 1.0 or 1.1, wherein the plant based gelling agent is palm kernel oil.
1.3 The personal care composition of any of Composition 1.0, 1.1, or 1.2, wherein the amount of the gelling agent (e.g., palm kernel oil) is from 25%-35% by wt of the total composition (e.g., about 27% by wt or about 33% by wt.).
1.4 Any of the preceding personal care compositions, wherein the structuring agent is selected from the group consisting of: Steareth-21, Steareth-10, Steareth-11, Steareth-1, Steareth-15, Steareth-20, Ceteareth-6, Ceteareth-7, Ceteareth-8, Ceteareth-9, Ceteareth-10, Ceteareth-11, and Ceteareth-12, polyglyceryl-6 palmitate/succinate mixture, glyceryl stearate, cetearyl alcohol (e.g., a 70/30 Stearyl/Cetyl Alcohol Blend), and combinations thereof.
1.5 The personal care composition of 1.4, wherein the structuring agent is cetearyl alcohol (e.g., a 70/30 Stearyl/Cetyl Alcohol Blend).
1.6 Any of the preceding personal care compositions, wherein the amount of the one or more structuring agent is from 15% by wt-25% by wt, wherein the weight is relative of the total composition.
1.7 Any of the preceding personal care compositions, wherein the amount of the one or more structuring agent is from 18% by wt-23% by wt, wherein the weight is relative of the total composition (e.g., about 20% by wt or about 22% by wt).
1.8 Any of the preceding personal care compositions, wherein the glyceryl-based emulsifier is glyceryl monostearate or glyceryl monolaurate.
1.9 The personal care composition of 1.8, wherein the glyceryl-based emulsifier is glyceryl monostearate.
1.10 The personal care composition of 1.8, wherein the glyceryl-based emulsifier is glyceryl monolaurate.
1.11 Any of the preceding personal care compositions, wherein the glyceryl-based emulsifier is present in an amount from 5% by wt.-20% by wt. of the total composition.
1.12 The personal care composition of 1.11, wherein the glyceryl-based emulsifier is present in amount from 5% by wt.-15 by wt. of the total composition (e.g., about 5% by wt.) (e.g., about 10% by wt.) (e.g., about 11 by wt.).
1.13 Any of the preceding personal care compositions in a cosmetically acceptable base suitable for application to the skin, e.g., a cosmetically acceptable base comprising one or more of water-soluble alcohols (such as C₂₋₈ alcohols including ethanol); glycols (including propylene glycol, dipropylene glycol, tripropylene glycol and mixtures thereof); glycerides (including mono-, di- and triglycerides); medium to long chain organic acids, alcohols and esters; surfactants (including emulsifying and dispersing agents); additional amino acids; structurants (including thickeners and gelling agents, for example polymers, silicates and silicon dioxide); emollients; fragrances; colorants (including dyes and pigments), and combinations thereof.
1.14 Any of the preceding personal care compositions, the metal-containing antiperspirant active ingredient contains aluminum, magnesium, strontium, zirconium, zinc or a combination thereof.
1.15 The personal care composition of 1.14, wherein the antiperspirant actives is selected from one of the following: aluminum chlorohydrate (ACH), aluminum chloride, aluminum chlorohydroxide, aluminum zirconium and combinations thereof.
1.16 Any of the preceding personal care compositions, wherein the metal-containing antiperspirant agent is aluminum zirconium tetrachlorohydrex glycine (ZAG) (e.g., activated ZAG).
1.17 Any of the preceding personal care compositions, wherein the metal-containing antiperspirant agent is the only active odor control ingredient (e.g., aluminum zirconium tetrachlorohydrex glycine is the only active odor control ingredient in the antiperspirant composition).
1.18 Any of the preceding personal care composition, wherein the metal-containing antiperspirant agent is present in an amount of 1 to 30% by weight of the composition, (e.g., from 10 to 30%, 11 to 25%, 12 to 20%, 13 to 15%, 14 to 20%, 15 to 20%, 11 to 15%, or 12 to 14% by weight of the composition)
1.19 The composition of any of the preceding personal care compositions, wherein the metal-containing antiperspirant agent comprises an amount of active ingredient present in an amount of 5%-25% by wt. of the composition (e.g., wherein the amount of active ingredient is about 5%) (e.g., wherein the amount of active ingredient is about 10%) (e.g., wherein the amount of active ingredient is about 20%).
1.20 The personal care composition of 1.18, wherein the metal-containing antiperspirant agent is aluminum zirconium tetrachlorohydrex glycine (ZAG) and is present in an amount of 12.5%-28% by wt. of the composition.
1.21 The personal care composition of any of compositions 1.14-1.20, wherein the amount of active ingredient in the aluminum zirconium tetrachlorohydrex glycine (ZAG) complex is from 5% by wt-25% by wt of the total composition (e.g., ZAG active present from 7%-22% by wt. of the total composition).
1.22 The personal care composition of 1.21, wherein the amount of the active is about 5 wt %, about 10 wt %, about 15 wt %, or about 20 wt %, by wt. of the total composition.
1.23 The composition of 1.22, wherein the active is ZAG (e.g., about 5 wt %, about 10 wt %, about 15 wt %, or about 20 wt %, by wt. of the total composition).
1.24 The personal care composition of 1.23, wherein the ZAG is activated.
1.25 Any of the preceding personal care compositions, further comprising a non-volatile emollient.
1.26 Any of the preceding personal care compositions, further comprising a second emollient source, wherein the emollient is selected from one or more of: PPG-14 butyl ether, PPG-3 myristyl ether, secondary alcohol ethoxylates, stearyl alcohol, stearic acid and salts thereof, glyceryl monoricinoleate, isobutyl palmitate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide, and combinations thereof.
1.27 Any of the preceding personal care compositions, wherein the first source of emollient comprises C₁₂₋₁₅ alkyl benzoate.
1.28 Any of the preceding personal care compositions, wherein the first source of emollient comprises mineral oil.
1.29 Any of the preceding personal care compositions, wherein the first source of emollient comprises caprylyl glycol.
1.30 Any of the preceding personal care compositions, wherein the first source of emollient comprises dimethicone.
1.31 Any of the preceding personal care compositions, wherein the first source of emollient comprises dicaprylyl ether.
1.32 Any of the preceding personal care compositions, wherein the first source of emollient comprises ethyl trisiloxane.
1.33 Any of the preceding personal care compositions, further comprising an additional volatile emollient.
1.34 Any of the preceding personal care compositions, further comprising an antioxidant selected from citric acid, butylated hydroxytoluene, pentaerythrityl tetra-di-t-butyl hydroxyhydrocinnamate.
1.35 The personal care composition of 1.34, wherein the antioxidant is present in an amount of about 0.1 to about 1 wt. %.
1.36 Any of the preceding personal care compositions, further comprising one or more inert fillers (e.g., configured to give specific sensorial feelings to the antiperspirant composition when applied.)
1.37 The personal care composition of 1.36, wherein the inert fillers is selected from: vegetable starches, talc, fumed silica and/or inorganic clays, polyethylene, and mixtures thereof.
1.38 The personal care composition of 1.37, wherein the inert filler comprises a tapioca starch, which is a starch extracted from cassava root. (e.g., a tapioca starch that is hydrophobically modified with polymethylsilsesquioxane).
1.39 Any of the preceding personal care composition, wherein the personal care composition comprises one or more of: fragrances, preservatives, antioxidants, colorants and combinations thereof.
1.40 Any of the preceding personal care compositions, wherein the personal care compositions is in the form selected from: antiperspirants, deodorants, body washes, shower gels, bar soaps, shampoo, hair conditioners, and cosmetics.
1.41 The personal care composition of 1.40, wherein the personal care composition is an antiperspirant.
1.42 Any of the preceding personal care compositions further comprising no greater than 4% of cyclomethicone, by wt. of the total composition (e.g., 0%-4% by wt.) (e.g., 0.1%-4% by wt.).
1.43 Any of the preceding personal care compositions, wherein the composition is substantially free of cyclomethicone (e.g., no greater than 0.1% by wt of the composition) (e.g., from 0.05-0.1% by wt.).
1.44 Any of the preceding personal care compositions, wherein the composition does not contain any cyclomethicone.
1.45 Any of the preceding personal care compositions, comprising:
   From 25%-40% by wt of palm kernel oil (e.g., about 27% PKO) (e.g., about 36% PKO)
   from 15% by wt-25% by wt, of a mixture of stearyl and cetyl alcohol (e.g., a 70/30 Stearyl/Cetyl Alcohol Blend);
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of ZAG active ingredient is 10% by wt or 20 by wt. of the total composition);
   A first source of emollients, wherein the emollients comprises one or more selected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt of the total composition (e.g., from 0.05%-14.9% by wt of the total composition); and wherein the composition (e.g., and antiperspirant) does not contain any cyclomethicone.
1.46 Any of the personal care compositions from 1.0-1.41, comprising:
   From 25%-40% by wt. of palm kernel oil (e.g., about 27% PKO) (e.g., about 36% PKO)
   from 15% by wt.-25% by wt of a mixture of stearyl and cetyl alcohol (e.g., a 70/30 Stearyl/Cetyl Alcohol Blend),
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   A first source of emollients, wherein the emollients comprise one or more selected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt. of the total composition (e.g., from 0.05%-14.9% by wt. of the total composition); and wherein the composition is an antiperspirant that does not contain more than 5% cyclomethicone by wt of the composition (e.g., from 0.1%-5% by wt. of cyclomethicone).
1.47 Any of the personal care compositions from 1.0-1.41, comprising:
   From 25%-40% by wt. of palm kernel oil (e.g., about 27% PKO) (e.g., about 36% PKO)
   from 15% by wt.-25% by wt., of a mixture of stearyl and cetyl alcohol (e.g., a 70/30 Stearyl/Cetyl Alcohol Blend);
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   A first source of emollients, wherein the emollients comprises one or more selected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt. of the total composition (e.g., from 0.05%-14.9% by wt. of the total composition); and wherein the composition (e.g., an antiperspirant) does not contain more than 1% cyclomethicone by wt of the composition (e.g., from 0.1%-1% by wt. of cyclomethicone).
1.48 Any of the personal care compositions from 1.0-1.41, comprising:
   From 25%-40% by wt of palm kernel oil (PKO) (e.g., about 27% PKO) (e.g., about 36% PKO)
   from 15% by wt-25% by wt, of a mixture of stearyl and cetyl alcohol (e.g., 70/30 Stearyl/Cetyl Alcohol Blend);
   from 5% by wt.-15 by wt. of either glyceryl monostearate or glyceryl monolaurate (e.g., about 10.5%);
   from 10 to 30%, by wt. of aluminum zirconium tetrachlorohydrex glycine (e.g., activated) (e.g., wherein the amount of ZAG active ingredient is 10% by wt or 20 by wt. of the total composition);
   from 5%-14% by wt. of a first source of emollients, wherein the emollients comprise C12-C15 alkyl benzoate and caprylyl glycol; and
wherein the composition (e.g., an antiperspirant) does not contain any cyclomethicone; wherein the weights are relative to the total weight of the composition.
1.49 Any of the personal care compositions from 1.0-1.41, comprising:
   From 25%-40% by wt. of palm kernel oil (PKO) (e.g., about 27% PKO) (e.g., about 36% PKO)
   from 15% by wt.-25% by wt., of a mixture of stearyl and cetyl alcohol (e.g., a 70/30 Stearyl/Cetyl Alcohol Blend);
   from 5% by wt.-15 by wt. of either glyceryl monostearate or glyceryl monolaurate (e.g., about 10.5%);
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   from 5%-14% of a first source of emollients, wherein the emollients comprise C12-C15 alkyl benzoate and caprylyl glycol; and
wherein the composition (e.g., an antiperspirant) does not contain more than 5% cyclomethicone by wt. of the composition (e.g., from 0.1%-5% by wt. of cyclomethicone);
wherein the weights are relative to the total weight of the composition.
1.50 Any of the personal care compositions from 1.0-1.41, comprising:
   From 25%-40% by wt. of palm kernel oil (PKO) (e.g., about 27% PKO) (e.g., about 36% PKO)
   from 15% by wt.-25% by wt, of a mixture of stearyl and cetyl alcohol (e.g., a 70/30 Stearyl/Cetyl Alcohol Blend);
   from 5% by wt.-15 by wt. of either glyceryl monostearate or glyceryl monolaurate (e.g., about 10.5%);
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   from 5%-14% by wt. of a first source of emollients, wherein the emollients comprise C12-C15 alkyl benzoate and caprylyl glycol; and
wherein the composition (e.g., an antiperspirant) does not contain more than 1% cyclomethicone by wt. of the composition (e.g., from 0.1%-1% by wt. of cyclomethicone);
wherein the weights are relative to the total weight of the composition.
1.51 Any of the preceding compositions, wherein the personal care composition is in the form selected from: antiperspirants, deodorants, body washes, shower gels, bar soaps, shampoo, hair conditioners, and cosmetics.
1.52 Any of the personal care compositions from 1.0-1.41, comprising:
   From 25%-40% by wt. of palm kernel oil (e.g., about 27% PKO) (e.g., about 36% PKO)
   from 15% by wt.-25% by wt., of a mixture of stearyl and cetyl alcohol (e.g., a 70/30 Stearyl/Cetyl Alcohol Blend);
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   A first source of emollients, wherein the emollient comprises one or more selected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt. of the total composition (e.g., from 0.05%-14.9% by wt. of the total composition); and
wherein the composition is an antiperspirant that does not contain any cyclomethicone;
wherein the weights are relative to the total weight of the composition.
1.53 Any of the preceding personal care compositions, comprising:
   From 25%-40% by wt. of palm kernel oil (e.g., about 27% PKO) (e.g., about 36% PKO)
   from 15% by wt.-25% by wt. of a 70/30 Stearyl/Cetyl end (e.g., about 20%-22% by wt.);
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of active ingredient is 10% by wt. or 20 by wt. of the total composition);
   a first source of emollients (e.g., from 5%-14%), wherein the emollient comprises, or consists of, C12-C15 alkyl benzoate and caprylyl glycol; and
wherein the amount of the first source of emollients does not exceed 15% by wt. of the total composition (e.g., from 0.05%-14.9% by wt. of the total composition); and
wherein the composition is an antiperspirant that does not contain any cyclomethicone;
wherein the weights are relative to the total weight of the composition.
1.54 Any of the personal care compositions from 1.0-1.41, comprising:
   From 25%-40% by wt. of palm kernel oil (e.g., about 27% PKO) (e.g., about 36% PKO)
   from 15% by wt.-25% by wt., of a 70/30 Stearyl/Cetyl Alcohol Blend (e.g., about 20%-22% by wt.);
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of active ingredient is 10% by wt. or 20 by wt. of the total composition);
   A first source of emollients (e.g., from 5%-14%), wherein the emollient comprises, or consists of C12-C15 alkyl benzoate and caprylyl glycol; and
wherein the amount of the first source of emollients does not exceed 15% by wt. of the total composition (e.g., from 0.05%-14.9% by wt. of the total composition); and
wherein the composition is an antiperspirant that contains cyclomethicone from 0.1%-5% by wt. of the composition;
wherein the weights are relative to the total weight of the composition.
1.55 Any of the preceding personal care compositions, comprising:
   From 25%-40% by wt. of palm kernel oil (e.g., about 27% PKO) (e.g., about 36% PKO)
   from 15% by wt.-25% by wt., of a mixture of stearyl and cetyl alcohol (e.g., a 70/30 Stearyl/Cetyl Alcohol Blend);
   from 5% by wt.-15 by wt. of either glyceryl monostearate or glyceryl monolaurate;
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   A first source of emollients, wherein the emollient comprises one or more elected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt. of the total composition (e.g., from 0.05%-14.9% by wt. of the total composition).
1.56 The composition of 1.55, wherein the composition is an antiperspirant that does not contain any cyclomethicone.
1.57 The composition of 1.55, wherein the composition is an antiperspirant that does not contain more than 5% cyclomethicone by wt. of the composition (e.g., from 0.1%-5% by wt. of cyclomethicone).
1.58 The composition of 1.55, wherein the composition is an antiperspirant that does not contain more than 1% cyclomethicone by wt. of the composition (e.g., from 0.1%-1% by wt. of cyclomethicone).
1.59 The composition of any of the preceding personal care compositions comprising:
   From 25%-40% by wt. of palm kernel oil (e.g., about 27% PKO) (e.g., about 36% PKO)
   from 15% by wt.-25% by wt., of a mixture of stearyl and cetyl alcohol (e.g., a 70/30 Stearyl/Cetyl Alcohol Blend);
   from 5% by wt.-15 by wt. of either glyceryl monostearate or glyceryl monolaurate (e.g., about 10.5%);
   from 10 to 30%, by wt. of aluminum zirconium tetrachlorohydrex glycine (e.g., activated) (e.g., wherein the amount of ZAG active ingredient is 10% by wt. or 20 by wt. of the total composition);
   A first source of emollients, wherein the emollient comprises C12-C15 alkyl benzoate and caprylyl glycol (e.g., from 10%-14% by wt. of the first source of emollients, relative to the total weight of the composition)
1.60 The personal care composition of 1.59, wherein the composition does not contain any cyclomethicone.
1.61 The personal care composition of any of the preceding compositions, wherein the amount of the first source of emollients is from 0.5%-14.9% by wt. of the total composition (e.g., from 5%-14.9%) (e.g., from 10%-14.9% by wt.).

As used herein, the term “activated”, for example, “activated aluminum zirconium tetrachlorohydrex glycine” or “activated ZAG”, refers to the metal-containing antiperspirant active ingredient that has been modified in order to contain a large component of the most active molecular species (i.e. K_{d}=0.4). As used herein, the term “active ingredient”, in connection with the metal containing antiperspirant agent, refers to the active ingredient that imparts antiperspirant benefit.

As used herein, the term “antiperspirant composition” is used to describe personal care compositions that function as antiperspirant compositions, deodorant compositions, or as dual purpose antiperspirant-deodorant compositions.

As used herein, the term “low cyclomethicone” or “low amounts of cyclomethicone” refers to compositions which contain no more than 5% by wt. of cyclomethicone, relative to the weight of the total composition (e.g., from 0.1-5% by wt.).

As used herein, the term antiperspirant can refer to any material that can form a “plug” in a pore to reduce sweating, or antiperspirant refers to those materials classified as antiperspirants by the Food and Drug Administration under 21 CFR part 350. Antiperspirants may also be deodorants, particularly in the case of the compositions of this disclosure, as the aluminum, magnesium, strontium, zirconium and zinc-containing active ingredients have antibacterial properties and can reduce odor-causing bacteria on the skin.

As used herein, “ZAG” refers to “aluminum zirconium tetrachlorohydrex glycine” which is a preparation that can be used as an antiperspirant in deodorant products.

As used herein, “personal care composition” is meant to include a composition for topical application to the skin of mammals, especially humans. The personal care composition is formulated into a product which can be applied to a human body for improving appearance, and/or cleansing and/or odor control and/or general aesthetics.

The antiperspirant active ingredients for use in the antiperspirant embodiments of the present invention include any compound, composition or other material having antiperspirant activity.

In one aspect, antiperspirant actives used in combination with any of the compositions of the disclosure, e.g., any of Composition 1.0 et seq, include astringent metallic salts, especially inorganic and organic salts of aluminum, zirconium and zinc, as well as mixtures thereof. In another aspect, actives used in combination with any of Composition 1.0 et seq include aluminum-containing and/or zirconium-containing salts or materials, such as aluminum halides, aluminum hydroxyhalides, zirconyl oxyhalides, zirconyl hydroxyhalides, and mixtures thereof. Especially useful antiperspirant actives suitable for use in the formulations include aluminum bromohydrate, aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum zirconium chlorohydrate, aluminum zirconium trichlorohydrate, aluminum zirconium tetrachlorohydrate, aluminum zirconium pentachlorohydrate, aluminum zirconium octachlorohydrate, aluminum zirconium tetrachlorohydrex propylene glycol complex, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, aluminum chloride, aluminum sulfate, buffered aluminum sulfate, potassium alum, sodium aluminum chlorohydroxy lactate and combinations thereof.

In one aspect, any of the compositions of the disclosure, e.g., any of Composition 1.0 et seq, can be any type of personal care composition. In certain embodiments, the composition is any composition in which it is desired to include an antibacterial agent for application to the skin. Examples of such compositions include, but are not limited to, personal care compositions, antiperspirants, deodorants, body washes, shower gels, bar soaps, shampoo, hair conditioners, and cosmetics.

In one aspect, any of the compositions of the disclosure, e.g., any of Composition 1.0 et seq, can be in the form of antiperspirant/deodorant compositions, and the carrier can be any carrier that is used for antiperspirants/deodorants. The carrier can be in the form of a stick, a gel, a roll-on, or an aerosol. For stick formulations, the carrier may include oils and/or silicones and gelling agents. An example of a formulation can be found in US2011/0076309A1, incorporated by reference herein.

In one aspect, optional ingredients that can be included in an antiperspirant and/or deodorant formulation of the compositions of the disclosure, e.g., any of Composition 1.0 et seq., include: solvents; water-soluble alcohols such as C₂₋₈ alcohols including ethanol; glycols including propylene glycol, dipropylene glycol, tripropylene glycol and mixtures thereof; glycerides including mono-, di- and triglycerides; medium to long chain organic acids, alcohols and esters; surfactants including emulsifying and dispersing agents; amino acids including glycine; structurants including thickeners and gelling agents, for example polymers, silicates and silicon dioxide; emollients; fragrances; and colorants including dyes and pigments. If desired, an antiperspirant and/or deodorant agent additional to the antiperspirant active ingredient can be included, for example an odor reducing agent such as a sulfur precipitating agent, e.g., copper gluconate, zinc gluconate, zinc citrate, etc.

In one aspect, the compositions of the disclosure, e.g., any of Composition 1.0 et seq, can also further comprise additional emollients in any desired amount to achieve a desired emollient effect. Emollients are known in the art and are used to impart a soothing effect on the skin. Non-volatile emollients are preferable. Classes of non-volatile emollients include non-silicone and silicone emollients. Non-volatile, non-silicone emollients include C₁₂₋₁₅ alkyl benzoate. The non-volatile silicone material can be a polyethersiloxane, polyalkyarylsiloxane or polyethersiloxane copolymer. An illustrative non-volatile silicone material is phenyl trimethicone. Non-limiting examples of emollients can be found in U.S. Pat. No. 6,007,799. Examples include, but are not limited to, PPG-14 butyl ether, PPG-3 myristyl ether, secondary alcohol ethoxylates (e.g. Tergitol sold by Dow Chemical Company, Midland, MI) stearyl alcohol, stearic acid and salts thereof, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide.

In one aspect, the compositions of the disclosure, e.g., any of Composition 1.0 et seq, can contain a fragrance. Any known fragrance can be used in any desired amount. In one embodiment, the amount of fragrance is 0.01 to 10 wt. %.

In one aspect, antioxidants may be added to the compositions of the disclosure, e.g., any of Composition 1.0 et seq, preferably to act as ingredient protectants and for maintenance of long-term stability of the composition. Examples of antioxidants include, but are not limited to citric acid, butylated hydroxytoluene, pentaerythrityl tetra-di-t-butyl hydroxyhydrocinnamate.

In one aspect, the compositions of the disclosure, e.g., any of Composition 1.0 et seq, may also contain polymeric materials for thickening, such as polyamides, cellulose derivatives (e.g., hydroxypropylcellulose, hydroxypropyl methyl cellulose, etc.) and natural or synthetic gums, such as polyglycerides including agar, agarose, pectin, or guars or mixtures or combinations thereof. One class of materials worthy of attention for thickening a water-immiscible phase comprises derivatives of hydrolysed starch or other polysaccharides, including in particular esterified dextrins, such as dextrin palmitate. A further class of polymers that can be included with any of C are particularly directed to structuring an oil phase containing a silicone oil comprises polysiloxane elastomers. Suspending agents such as silicas or clays such as bentonite, montmorillonite or hectorite, including those available under the trademark Bentone can also be employed to thicken liquid compositions according to the invention. The composition can be thickened with non-polymeric organic gellants, including selected dibenzylidene alditols (e.g., dibenzylidene sorbitol).

In one aspect, any of the antiperspirant/deodorant compositions of the disclosure, e.g., any of Composition 1.0 et seq., can be applied to axillary areas to reduce sweat and/or odor. The compositions can be applied by hand or via their packaging.

In still another aspect, the present disclosure relates to a method of administering any of Composition 1.0 et seq for prophylaxis or reduction of skin irritation, a method for treatment of skin irritation, a method for reducing, eliminating or suppressing the irritating, preferably the skin-irritating, action of a substance or substance mixture, and a kit comprising (i) a formulation, a cosmetic product or a pharmaceutical product according to the present invention and, spatially separated, (ii) one or more substances or substance mixtures having an irritating, preferably a skin-irritating, action.

In another aspect, any of the compositions of the disclosure, e.g., any of Composition 1.0 et seq, can be an antiperspirant composition that is formulated into topical antiperspirant and/or deodorant formulations suitable for application to skin, illustratively a stick, a gel, a cream, a roll-on, a soft solid, a powder, a liquid, an emulsion, a suspension, a dispersion or a spray. Any of Composition 1.0, et seq can comprise a single phase or can be a multi-phase system, for example a system comprising a polar phase and an oil phase, optionally in the form of a stable emulsion. The composition can be liquid, semi-solid or solid. The antiperspirant and/or deodorant formulation can be provided in any suitable container such as an aerosol can, tube or container with a porous cap, roll-on container, bottle, container with an open end, barrel, etc.

In one aspect, the compositions of the disclosure, e.g., any of Composition 1.0 et seq, can be used in a method to reduce sweating by applying the composition to skin. In certain embodiments, the application is to axilla. Also, the compositions can be used to kill bacteria by bringing the bacteria into contact with the composition.

Thus the disclosure provides (i) a method for controlling perspiration comprising applying to skin an antiperspirant effective amount of a formulation of any embodiment embraced or specifically described herein, e.g., any of Compositions 1 et seq.; and (ii) a method for controlling odor from perspiration comprises applying to skin a deodorant effective amount of a formulation of any embodiment embraced or specifically described herein, e.g., any of Compositions 1 et seq.

In one aspect, any of Composition 1.0 et seq can be used in a method of reducing or diminishing yellowing on a user's fabric (e.g., clothing), wherein any of Composition 1.0 et seq is applied to the skin of the user and wherein the use or application of any of Composition 1.0 et seq reduces or diminishes the amount of yellowing on the user's fabric relative to an antiperspirant comprising cyclomethicone (e.g., in amounts of 5% by wt or greater), and wherein the antiperspirant comprising cyclomethicone does not comprise a glyceryl-based emulsifier (e.g., glyceryl monostearate).

The compositions and formulations as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total amount of the compositions. The amounts given are based on the active weight of the material.

Unless otherwise specifically identified, the ingredients for use in the compositions and formulations of the present invention are preferably cosmetically acceptable ingredients. By “cosmetically acceptable” is meant suitable for use in a formulation for topical application to human skin. A cosmetically acceptable excipient, for example, is an excipient which is suitable for external application in the amounts and concentrations contemplated in the formulations of this invention, and includes for example excipients which are “Generally Recognized as Safe” (GRAS) by the United States Food and Drug Administration.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1: Representative Antiperspirant Formula

TABLE 1

| Material | Wt % |
|---|---|
| PALM KERNEL OIL | 25-35% |
| STRUCTURING AGENT | 15-25% |
| ZAG | 15-25% |
| (″aluminum zirconium | |
| tetrachlorohydrex | |
| glycine″) | |
| C12-15 Alkyl benzoate | 10-14% |
| Glyceryl monostearate | 5-15% |
| FRAGRANCE | 1.0-2.0% |
| Total | 100% |

### Example 2: Comparison of Antiperspirant Formulas that are “Cyclomethicone Free” or “Low Cyclomethicone”

The active level is essentially controlled for in the below antiperspirant stick formulations, i.e., the range of active ZAG (“aluminum zirconium tetrachlorohydrex glycine”) is between 15.83% and 16.07%, by wt. of the composition. At approximately the same active level, the formula performed better than the Soy/PKO base with partially activated aluminum-zirconium tetrachlorohydrex gly and higher levels (14.9%) of cyclomethicone. Certain market formulations incorporate a Soy/PKO base, with higher levels of cyclomethicone, and a metal-containing active. Table 2 illustrates that reformulation of the base, and to also lower or reduce the amount of cyclomethicone, has the unexpected benefit of maintaining, or even improving, performance of the antiperspirant. The “% reduction vs. untreated site” refers to the relative reduction in sweat compared to an untreated site. In this assay, the two market control formulas—Market Control A and Market Control B—demonstrated 68.7% and 42.7% reduction vs. untreated site, respectively. Note Control A is a market antiperspirant containing 20% aluminum-zirconium tetrachlorohydrex gly:

TABLE 2

| | % Reduction | | % Effective | | |
|---|---|---|---|---|---|
| | VS. | Statistical | active (by | | |
| | Untreated | Group | wt. of the | | |
| Product | Site | (p < 0.05) | composition) | Composition (% by wt.) | |
| Formula | 58.1 | b | 15.9 | PKO | 33% |
| 1 | | | ZAG | Stearyl/cetyl alcohol | 22% |
| | | | Active | C12-15 Alkyl benzoate | 12.4% |
| | | | | Glyceryl monostearate | 11% |
| | | | | Dimethicone | 4% |
| | | | | Mineral oil | 2% |
| | | | | Caprylyl glycol | 1% |
| Formula | 57.0 | b, c | 15.8 | PKO | 33% |
| 2 | | | ZAG | Stearyl/cetyl alcohol | 22% |
| | | | Active | C12-15 Alkyl benzoate | 12.4% |
| | | | | Glyceryl monostearate | 11% |
| Formula | 54.2 | b, c | 15.9 | PKO | 33% |
| 3 | | | ZAG | Stearyl/cetyl alcohol | 22% |
| | | | Active | C12-15 Alkyl benzoate | 12.4% |
| | | | | Glyceryl monostearate | 11% |
| | | | | Cyclomethicone | 3% |
| Formula | 53.4 | b, c | 16.1 | PKO | 33% |
| 4 | | | ZAG | Stearyl/cetyl alcohol | 22% |
| | | | Active | C12-15 Alkyl benzoate | 12.4% |
| | | | | Glyceryl monostearate | 11% |
| | | | | Dimethicone | 4% |
| | | | | Cyclomethicone | 4% |
| | | | | Caprylyl glycol | 1% |
| | | | | Mineral oil | 4% |
| Formula | 53.3 | b, c | 16 | PKO | 33% |
| 5 | | | ZAG | Stearyl/cetyl alcohol | 22% |
| | | | Active | C12-15 Alkyl benzoate | 12.4% |
| | | | | Glyceryl monostearate | 11% |
| | | | | Cyclomethicone | 8% |
| | | | | Mineral oil | 4% |
| Formula | 53.1 | b,c | 16.1 | PKO | 33% |
| 6 | | | ZAG | Stearyl/cetyl alcohol | 22% |
| | | | Active | C12-15 Alkyl benzoate | 12.4% |
| | | | | Glyceryl monostearate | 11% |
| | | | | Dimethicone | 8% |
| | | | | Cyclomethicone | 4% |
| | | | | Caprylyl glycol | 1% |
| Formula | 52.1 | b, c | 15.9 | PKO | 33% |
| 7 | | | Active | Stearyl/cetyl alcohol | 22% |
| | | | ZAG | C12-15 Alkyl benzoate | 12.4% |
| | | | | Glyceryl monostearate | 11% |
| | | | | Dimethicone | 3% |
| Formula | 40.8 | d | 15.8 | PKO | 23.3% |
| 9 | | | Partially activated | Stearyl alcohol | 15.5% |
| | | | Aluminum- | Cyclomethicone | 14.9% |
| | | | Zirconium | C12-15 Alkyl benzoate | 5.9% |
| | | | Tetrachloro- | Castor oil | 5.5% |
| | | | hydrexgly | PEG-8 Distearate | 3.2% |
| | | | | PPG-14 Butyl Ether | 5.9% |
| | | | | Soybean oil | 3.3% |
| | | | | Behenyl Alcohol | 0.2% |
| | | | | Citric Acid | 0.04% |
| | | | | Tinogard | 0.02% |

### Example 3: Comparison of Antiperspirant Formulas that are “Cyclomethicone Free” or “Low Cyclomethicone”

The sweat reduction efficacy of antiperspirant sticks are tested where the level of activated ZAG (“aluminum zirconium tetrachlorohydrex glycine”) is at 20% by wt., 15% by wt. and 10% by wt (all relative to the total weight of the composition). As demonstrated in Table 3, core formulas with levels of active at 15% by wt., can achieve values of sweat reduction similar to the Market Formula A antiperspirant stick (high efficacy control). In general, the formula with 20% active ingredient perform better in terms of percent reduction sweat reduction, than the Market Formula A, high efficacy control. Similar to the above Table 2, Table 3 illustrates that reformulation of the base, and to also lower or reduce the amount of cyclomethicone, has the unexpected benefit of maintaining, or even improving, performance of the antiperspirant. The “% reduction vs. untreated site” refers to the relative reduction in sweat compared to an untreated site. Market Control A and Market Control B—demonstrated 47.1% and 25.1% reduction vs. untreated site, respectively.

TABLE 3

| | % | | | | |
|---|---|---|---|---|---|
| | Reduction | | % | | |
| | VS. | Statistical | Effective | | |
| | Untreated | Group | active | | |
| Product | Site | (p < 0.05) | (by wt.) | Composition | |
| Formula | 50.2 | a | 20% by | PKO | 27% |
| 10 | | | wt. ZAG | Stearyl/cetyl alcohol | 18.3% |
| | | | Active | C12-15 Alkyl benzoate | 12.4% |
| | | | | Glyceryl monostearate | 11% |
| | | | | Castor Wax | 4% |
| Formula | 47.7 | a, b | 15% by | PKO | 25% |
| 11 | | | wt. ZAG | Stearyl/ cetyl alcohol | 22.8% |
| | | | Active | C12-15 Alkyl benzoate | 6.6% |
| | | | | Glyceryl monostearate | 19% |
| | | | | Petrolatum | 5% |
| Formula | 44.2 | a, b, c, d | 15% by | PKO | 33% |
| 12 | | | wt. ZAG | Stearyl/ cetyl alcohol | 18% |
| | | | Active | C12-15 Alkyl benzoate | 12.4% |
| | | | | Glyceryl monostearate | 11% |
| | | | | Castor Wax | 4% |
| Formula | 40.8 | b, c, d,e | 15% by | PKO | 32% |
| 13 | | | wt. ZAG | Stearyl/ cetyl alcohol | 17% |
| | | | Active | C12-15 Alkyl benzoate | 12.4% |
| | | | | Glyceryl monostearate | 11% |
| | | | | Castor Wax | 4% |
| | | | | Corn Starch | 2% |
| Formula | 40.4 | c, d,e | 15% by | PKO | 33% |
| 14 | | | wt. ZAG | Stearyl/ cetyl alcohol | 22% |
| | | | Active | C12-15 Alkyl benzoate | 12.4% |
| | | | | Glyceryl monostearate | 11% |
| Formula | 39.6 | d, e | 10% by | PKO | 36.8% |
| 15 | | | wt. ZAG | Stearyl/ cetyl alcohol | 22% |
| | | | Active | C12-15 Alkyl benzoate | 12.4% |
| | | | | Glyceryl monostearate | 11% |
| | | | | Castor Wax | 4% |
| Formula | 38.5 | d, e, f | 10% by | PKO | 36.3% |
| 16 | | | wt. ZAG | Stearyl/ cetyl alcohol | 21.5% |
| | | | Active | C12-15 Alkyl benzoate | 12.4% |
| | | | | Glyceryl monostearate | 11% |
| | | | | Castor Wax | 4% |
| | | | | Stearic Acid | 1% |
| Formula | 34.4 | e, f | 10% by | PKO | 35.8% |
| 17 | | | wt. ZAG | Stearyl/ cetyl alcohol | 21% |
| | | | Active | C12-15 Alkyl benzoate | 12.4% |
| | | | | Glyceryl monostearate | 11% |
| | | | | Castor Wax | 6% |
| Formula | 30.8 | f, g | 10% by | PKO | 33% |
| 18 | | | wt. ZAG | Stearyl/ cetyl alcohol | 20.8% |
| | | | Active | C12-15 Alkyl benzoate | 12.4% |
| | | | | Glyceryl monostearate | 11% |
| | | | | Castor Wax | 4% |
| | | | | Cyclomethicone | 5% |

A clinical study is also done to test the efficacy of 18%, 10% and 8% (by wt.) active ingredient. Market Control A (Positive Control) and Market Control B—demonstrate 73.5% and 53.5% reduction vs. untreated site, respectively. ZAG used below refers to “aluminum zirconium tetrachlorohydrex glycine”.

TABLE 4

| | % Reduction | | % | | |
|---|---|---|---|---|---|
| | VS. | Statistical | Effective | | |
| | Untreated | Group | active | | |
| Product | Site | (p < 0.05) | (by wt.) | Composition | |
| Formula | 69.0 | b | 18% by | PKO | 31% |
| 19 | | | wt. ZAG | Stearyl/cetyl alcohol | 19.6% |
| | | | Active | C12-15 Alkyl benzoate | 7.0% |
| | | | | Glyceryl monostearate | 9.8% |
| | | | | Castor Wax | 2.6% |
| | | | | Corn Starch | 5% |
| Formula | 66.2 | b, c | 18% by | PKO | 31% |
| 20 | | | wt. ZAG | Stearyl/cetyl alcohol | 19.6% |
| | | | Active | C12-15 Alkyl benzoate | 11.0% |
| | | | | Glyceryl monostearate | 9.8% |
| | | | | Castor Wax | 3.6% |
| Formula | 62.8 | c, d | 10% by | PKO | 35.6% |
| 21 | | | wt. ZAG | Stearyl/cetyl alcohol | 22.4% |
| | | | Active | C12-15 Alkyl benzoate | 12.6% |
| | | | | Glyceryl monostearate | 11.2% |
| | | | | Castor Wax | 4.1% |
| Formula | 62 | c, d | 8% by | PKO | 36.8% |
| 22 | | | wt. ZAG | Stearyl/cetyl alcohol | 19.6% |
| | | | Active | C12-15 Alkyl benzoate | 13.1% |
| | | | | Glyceryl monostearate | 11.6% |
| | | | | Castor Wax | 4.2% |
| Formula | 57.2 | d, e | 10% by | PKO | 35.6% |
| 23 | | | wt. ZAG | Stearyl/cetyl alcohol | 22.4% |
| | | | Active | C12-15 Alkyl benzoate | 10.6% |
| | | | | Glyceryl monostearate | 11.2% |
| | | | | Castor Wax | 3.1% |
| | | | | Petrolatum | 3.0% |
| Formula | 54.9 | e, f | 10% by | PKO | 35.6% |
| 24 | | | wt. ZAG | Stearyl/cetyl alcohol | 22.4% |
| | | | Active | C12-15 Alkyl benzoate | 12.6% |
| | | | | Glyceryl monostearate | 11.2% |
| | | | | Castor Wax | 4.1% |
| Formula | 54.1 | d, e, f | 8% by | PKO | 36.8% |
| 25 | | | wt. ZAG | Stearyl/cetyl alcohol | 23.2% |
| | | | Active | C12-15 Alkyl benzoate | 11.1% |
| | | | | Glyceryl monostearate | 11.6% |
| | | | | Castor Wax | 3.21% |
| | | | | Petrolatum | 3% |
| Formula | 52.1 | e, f | 10% by | PKO | 35.6% |
| 26 | | | wt. ZAG | Stearyl/cetyl alcohol | 22.4% |
| | | | Active | C12-15 Alkyl benzoate | 8.6% |
| | | | | Glyceryl monostearate | 11.2% |
| | | | | Castor Wax | 3.1% |
| | | | | Corn Starch | 5% |
| Formula | 50.4 | g | 8% by | PKO | 36.8% |
| 27 | | | wt. ZAG | Stearyl/cetyl alcohol | 23.2% |
| | | | Active | C12-15 Alkyl benzoate | 9.1% |
| | | | | Glyceryl monostearate | 11.6% |
| | | | | Castor Wax | 3.2% |
| | | | | Corn Starch | 5% |

### Example 4: Representative Antiperspirant Formulations

| | Formula C | Formula D |
|---|---|---|
| | (20% by wt. | (10% by wt. |
| | ZAG Active) | ZAG Active) |
| Aluminum. | 27.3 | 14.0 |
| Zirconium. Tetrachlorohydrex- | | |
| GLY, (Low Fe) | | |
| PALM KERNAL OIL | 27.3 | 36.1 |
| FIRST SOURCE OF | 11.5 | 12.6 |
| EMOLLIENT | | |
| HYDROGENATED | 3.7 | 4.1 |
| CASTOR OIL | | |
| STRUCTURING AGENT | 20.2 | 22.2 |
| GLYCERYL | 10.1 | 11.1 |
| MONOSTEARATE | | |
| DISCLORING STABILIZER | 0.018 | 0.02 |
| Total | 100 | 100 |

### Example 5: Protection Against Yellowing on Fabric

Aluminum salts can result in yellow stain formation on cloths. Without being bound by theory, formulas of this example use an emulsifying agent (Glyceryl Monoesterate) to enhance the wash off process of the formula from the skin but also from fabric. By contrast, certain antiperspirants being wax based are hydrophobic which results in the water being repelled. The use of detergents to wash the fabric can increase the removal of it. However, the appearance of yellowing can still result from a mixture of sweat, sebum and the antiperspirant product that stays in the fabric and through uses it causes a buildup that increase the intensity of the yellow stains. Again, without being bound by theory, adding glyceryl monosterate provides a less hydrophobic material, and may actually diminish the amount of yellowing on fabric.

Here, Formulas C and D are subject to a simulated use test method to evaluate the impact of yellow staining of antiperspirant formulas on fabric. The amount of yellowing on fabric is determined by a colorimeter along a “b axis”, with a more positive number indicating more yellow.

Formulas C (described in Example 4 above) demonstrate that a high content of active ingredient (20%) can perform at parity to the Control. And Formula D (described in Example 4 above), which contains 10% active ingredient, demonstrates less yellow stains when compared to a Control with the same amount of active (i.e., 10%), demonstrating a difference of about 0.5 “b value” lower.

Control Formula Used as a Comparison:

| Ingredient | Amount (% by wt.) |
|---|---|
| Aluminum. | 14.4 |
| Zirconium. | (10% by wt. |
| Tetrachlorohydrex- | of active) |
| GLY, | |
| Plant-Based Gelling | 42 |
| Agent(s) | |
| Cyclomethicone | 10.9 |
| First Source | 17.2 |
| of Emollient | |
| Second Source | 4.3 |
| of Emollient | |
| Antioxidant | 0.08 |
| Synthetic Wax | 11.1 |
| Total | 100 |

While the present invention has been described with reference to embodiments, it will be understood by those skilled in the art that various modifications and variations may be made therein without departing from the scope of the present invention as defined by the appended claims.

1. A personal care composition comprising:
a. from 25%-45% by wt. of the total composition of a plant-based gelling agent
b. from 15%-30% by wt. of the total composition of one or more structuring agents
c. a glyceryl-based emulsifier;
d. a metal containing antiperspirant agent;
e. a first source of emollients, wherein the emollient comprises one or more elected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt of the total composition.

2. The personal care composition of claim 1, wherein the plant-based gelling agent is selected from the group consisting of: waxes, esters of fatty acid and fatty alcohol, triglycerides, partially or fully hydrogenated soybean oil, partially or fully hydrogenated castor oil, other partial or fully hydrogenated plant oils, or other cosmetically acceptable materials (e.g., which are solid or semi solid at room temperature and provide a consistency suitable for application to the skin) and combinations thereof.

3. The personal care composition of claim 1, wherein the plant-based gelling agent is palm kernel oil.

4. The personal care composition of claim 1, wherein the structuring agent is selected from the group consisting of: Steareth-21, Steareth-10, Steareth-11 Steareth-13, Steareth-15, Steareth-20, Ceteareth-6, Ceteareth-7, Ceteareth-8, Ceteareth-9, Ceteareth-10, Ceteareth-11, and Ceteareth-12, polyglyceryl-6 palmitate/succinate mixture, glyceryl stearate, cetearyl alcohol, and combinations thereof.

5. The personal care composition of claim 4, wherein the one or more structuring agent is cetearyl alcohol (e.g., a 70/30 Stearyl/Cetyl Alcohol Blend).

6. The personal care composition of claim 5, wherein the amount of the one or more structuring agent is from 18% by wt-23% by wt, wherein the weight is relative of the total composition.

7. The personal care composition of claim 1, wherein the glyceryl-based emulsifier is glyceryl monostearate or glyceryl monolaurate.

8. The personal care composition of claim 7, wherein the glyceryl-based emulsifier is glyceryl monostearate.

9. The personal care composition of claim 7, wherein the glyceryl-based emulsifier is glyceryl monolaurate.

10. The personal care composition of claim 1, wherein the glyceryl-based emulsifier is present in an amount from 5% by wt.-20% by wt. of the total composition.

11. The personal care composition of claim 1, wherein the metal-containing antiperspirant active ingredient contains aluminum, magnesium, strontium, zirconium, zinc or a combination thereof, optionally wherein the antiperspirant active ingredient is aluminum zirconium tetrachlorohydrex glycine (ZAG) present in an amount of 1 to 30% by weight of the composition.

12. (canceled)

13. (canceled)

14. The composition of claim 1, wherein the active is ZAG and the ZAG is present in an amount from 5%-25% by wt. of the total composition.

15. The composition of claim 14, wherein the ZAG is activated.

16. The personal care composition of claim 1, wherein the first source of emollient comprises C₁₂₋₁₅ alkyl benzoate, mineral oil, caprylyl glycol, dimethicone, dicaprylyl ether, or ethyl trisiloxane.

17. (canceled)

18. (canceled)

19. (canceled)

20. (canceled)

21. (canceled)

22. The personal care composition of claim 1, wherein the personal care compositions is in the form selected from: antiperspirants, deodorants, body washes, shower gels, bar soaps, shampoo, hair conditioners, and cosmetics.

23. (canceled)

24. The personal care composition of claim 1, wherein the composition is an antiperspirant does not contain any cyclomethicone.

25. The personal care composition of claim 1, wherein the composition is an antiperspirant that does not contain more than 5% cyclomethicone by wt. of the composition (e.g., 0.1%-5% by wt.).

26. The personal care composition of claim 1, comprising:
from 25%-40% by wt of palm kernel oil
from 15% by wt-25% by wt, of a mixture of stearyl and cetyl alcohol;
from 5% by wt.-15 by wt. of either glyceryl monostearate or glyceryl monolaurate;
from 10 to 30%, by wt. of aluminum zirconium tetrachlorohydrex glycine;
a first source of emollients, wherein the emollient comprises one or more elected from: C12-C15 alkyl benzoate, mineral oil, caprylyl glycol, dimethicone, dicaprylyl ether, ethyl trisiloxane and combinations thereof; and
wherein the amount of the first source of emollients does not exceed 15% by wt of the total composition (e.g., from 0.05%-14.9% by wt of the total composition).

27. The personal care composition of claim 26, wherein the composition is an antiperspirant composition that does not contain any cyclomethicone.

28. A method of reducing or diminishing yellowing on a user's fabric, wherein any of the personal care composition of claim 1 is applied to the skin of the user, and wherein the use or application of the personal care composition reduces or diminishes the amount of yellowing on the user's fabric relative to an antiperspirant comprising cyclomethicone (e.g., in amounts of 5% by wt. or greater); and wherein the antiperspirant comprising cyclomethicone does not comprise a glyceryl-based emulsifier (e.g., glyceryl monostearate).
